(19) United States
(12) Patent Application Publication
(10) Pub. No.: US 2009/0259253 A1
(43) Pub. Date: Oct. 15, 2009

(54) APPARATUS FOR STRETCHING THE VERTEBRAL COLUMN OF A PERSON

(75) Inventor: Christophe Bensoussan, Rosporden (FR)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: SATISFORM, Mellac (FR)

(21) Appl. No.: 12/374,370

(22) PCT Filed: Jul. 9, 2007
(86) PCT No.: PCT/FR07/01175

§ 371 Date: Jan. 19, 2009

(30) Foreign Application Priority Data
Jul. 19, 2006 (FR) 06.06594

(51) Int. Cl. A61F 5/00

(52) U.S. Cl. 606/242

(57) ABSTRACT

An apparatus for stretching the vertebral column of a person, comprising a table equipped with a traction system having fixing means for connecting a cable to the person's feet, and traction means acting on the cable to effect traction on the legs in order to stretch the legs and the column, and oscillating means that act on the cable so as to obtain movements of lateral oscillation of both legs, the traction means and the oscillating means adapted to be controlled by a control unit in order to combine movements of lateral oscillation and movements of traction. The apparatus can be used to effect movements of lateral oscillation in order to achieve muscle relaxation in the region of the pelvis and the lumbar region, and thereby make the subsequent stretching of the lower back region more effective.

### PRIORITY CLAIM

The present application is a National Phase entry of PCT Application No. PCT/FR2007/001175, filed Jul. 9, 2007, which claims priority from French Application Number 06.06594, filed Jul. 19, 2006, the disclosures of which are hereby incorporated by reference herein in their entirety.

### TECHNICAL FIELD

The present invention relates to an apparatus for stretching the vertebral column of a person, particularly the lower back, in order to soothe and/or prevent back aches. More particularly, the present invention relates to an apparatus for relaxing the pelvis and for an effective stretching of the lower back.

### BACKGROUND ART

U.S. Pat. No. 5,730,706 discloses a stretching and traction apparatus comprising a table for supporting a person lying on the back, and a traction system comprising at least one cable, fixing means for connecting the cable to the person's feet and traction means acting on the cable to effect traction on the legs in order to stretch the legs and the vertebral column. A person lying down on the table with his legs suspended tends to contract, thus greatly limiting the effect sought by the stretching operation. In order to prepare the person for the stretching operation, heating means integrated with the table are provided for heating the muscles surrounding the column and thus to try to reduce the tensions at these muscles before and during the stretching. Such localized heating proves to be difficult to implement. Moreover, with the person remaining static, such heating does not allow for an efficient relaxation of the pelvis and thus only has little impact on the effectiveness of the stretching.

### SUMMARY OF THE INVENTION

The present invention provides a new apparatus to overcome the aforementioned drawbacks, particularly allowing an efficient stretching of the lower back.

To this end, the present invention relates to an apparatus for stretching the vertebral column of a person, particularly the lower back. An embodiment of the invention comprises a table for supporting a person lying on the back, which table is provided with a traction system comprising at least one cable, fixing means for connecting the cable to the person's feet and traction means acting on the cable to effect traction on the legs in order to stretch the legs and the vertebral column, the apparatus further including oscillating means acting on the cable in order to obtain lateral oscillation movements of both legs on either side of the axis of the person's vertebral column, the traction means and the oscillating means being controllable by a control unit in order to combine movements of lateral oscillation of the legs and traction movements on the legs.

According to an embodiment of the invention, the apparatus comprises oscillating means for obtaining movements of lateral oscillation of the legs. In particular, the apparatus may be used to effect lateral oscillation movements in order to achieve muscle relaxation in the region of the pelvis and the lumbar region and thereby make the subsequent stretching more effective, especially in the lower back region. The apparatus according to an embodiment of the invention further provides a relaxing effect and a feeling of well being. Thus, according to one embodiment, the traction means and the oscillating means are controllable by the control unit to effect movements of lateral oscillation prior to one or more traction movements.

According to another feature of an embodiment of the invention, the traction means and the oscillating means are controllable by the control unit to simultaneously effect movements of lateral oscillation of the legs and one or more traction movements for stretching the lower back, preferably after having effected movements of lateral oscillation only. Thus, after movements of lateral oscillation of the legs for preparing the stretching, the stretching can be effected with or without oscillation.

According to an embodiment, the traction system comprises at least one pulley, mounted on a crossbar which is transversally assembled to the table, and whereon the cable is passed, the oscillating means being able to transversally move the pulley on the crossbar in order to effect the movements of lateral oscillation. The oscillating means may comprise a cam system fitted between the drive shaft of a motor and the pulley for transforming the shaft's rotational movement into a back-and-forth movement of the pulley on either side of the table's longitudinal plane, the motor being rotationally controlled by the control unit.

According to an embodiment, the apparatus comprises two cables, the traction system thus comprising two pulleys mounted on a same crossbar transversally assembled to the table, with a cable passing on each pulley, and the oscillating means adapted to transversally move the pulleys on the crossbar. In this case, the fixing means advantageously comprise a bar to which the cables are connected, the person's feet being assembled to the bar between the fixing points of the two cables on the bar, the spacing between the fixing points substantially corresponding to that of the two pulleys on the crossbar.

According to another feature, the apparatus further comprises lifting means for lifting the legs at an angle with respect to the column's axis during the oscillation movements and the traction movement(s).

According to an embodiment, the traction system comprises two posts fixed to the table supporting the crossbar above the table, and possibly an oscillation motor, the traction means, formed for example by a winch, thus serving as lifting means for the legs. Advantageously, the apparatus thus comprises an angle sensing system for sensing the cable's angle between the pulley and the fixing means, the sensing system being connected to the control unit for controlling the traction means and the oscillating means.

Advantageously, the oscillating means are controllable by the control unit, and/or modifiable, in order to vary the frequency, amplitude and/or duration of the oscillation movements, particularly according to the person's height, weight, age and/or gender, and/or the traction means are controllable by the control unit, and/or modifiable, in order to vary the duration and/or the traction force of each traction movement, and/or the successive number of traction movements, particularly according to the person's height, weight, age and/or gender.

### BRIEF DESCRIPTION OF THE DRAWINGS

The invention will be better understood, and other objects, details, characteristics and advantages will become more apparent in the following detailed explanatory description of a currently-preferred particular embodiment of the invention, with reference to the accompanying schematic drawings, in which:

FIG. 1 depicts a schematic perspective view of an apparatus according to an embodiment of the invention for particularly relaxing the pelvis then stretching the lower back;

FIGS. 2 and 3 respectively represent a side view and a rear view of the apparatus of the drawing free from the protective casings;

FIG. 4 is an enlarged perspective view of an upper part of the apparatus of FIG. 1, with no casing, illustrating the oscillation system;

FIG. 5 is a perspective view of a lower part of the apparatus of FIG. 1, with no casing, particularly showing, the traction means;

FIG. 6 depicts an enlarged partial perspective view of FIG. 4;

FIG. 7 depicts a perspective view of FIG. 6 seen from another view angle; and,

FIG. 8 is an enlarged perspective view of a front part of the apparatus of FIG. 1, depicting the armpit strapping system.

### DETAILED DESCRIPTION OF THE DRAWINGS

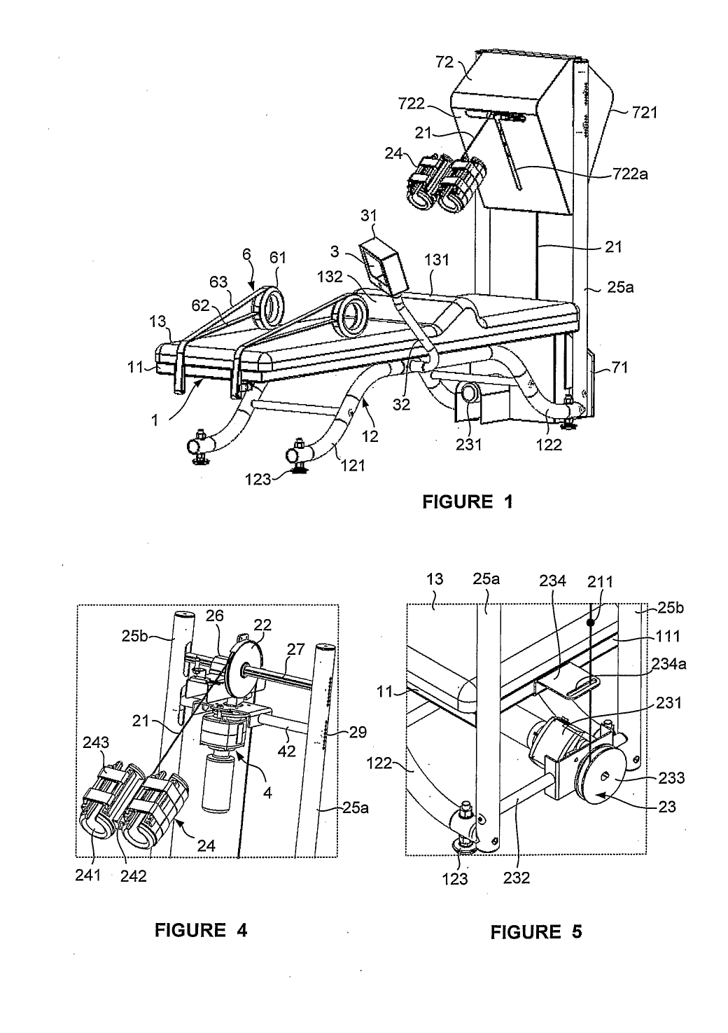

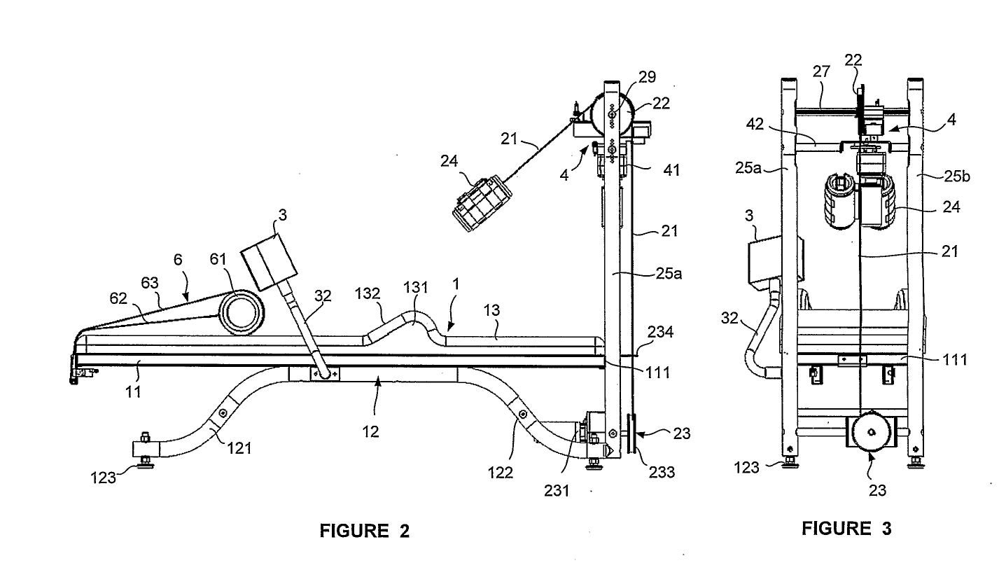

With reference to FIGS. 1 to 3, the apparatus according to an embodiment of the invention comprises a table 1 comprising a tray or horizontal frame 11 supported substantially horizontally on a chassis 12. The chassis comprises two front legs 121 and two rear legs 122 at the end of which height-adjustable skids 123 are provided, whereby the chassis rests on the ground. The upper tray is equipped with a mattress 13 comprising a transversal protrusion 131 which defines a bearing surface 132 for a person's buttocks.

The apparatus comprises a traction system for carrying out traction on the legs for extending the legs and back, by means of at least one cable-type link fixed to the legs. The traction system includes a cable 21 passing over an upper pulley 22 arranged above the rear end of the table. The cable is fixed by a first end to a winch 23 and by a second end to a fixing system 24 for connecting the cable to both legs of a person. The pulley is mounted between two vertical posts 25a, 25b assembled to the ends of the rear legs 122 of the chassis and to the rear edge 111 of the tray. The pulley is mounted moveable in translation between the two posts for enabling the movements of lateral oscillation as described hereinafter. In the present embodiment according to FIGS. 4 and 7, the pulley is mounted in a freely rotating manner on a slider 26. This slider is slidingly mounted on a splined horizontal tubular crossbar 27 that is assembled between the two posts, the slider comprising ribs housed in the longitudinal splines of the crossbar to ensure its rotational blocking.

The fixing system comprises two leg receiving chutes 241 parallely connected together by a central bracket 242 to which the cable 21 is fixed. Each chute is equipped with two straps 243 for tightening of the chute around a leg, above the foot, at the ankle and calf.

According to FIG. 5, the winch 23 comprises a motor 231 assembled to a supporting crossbar 232 mounted between the two posts, under the tray 11. The drive shaft of the motor, arranged horizontally and perpendicularly to the posts, bears a drum 233 whereon the cable 21 is fixed and wound. The winch motor is controlled by a parametrizable control unit, and can drive the drum in a first direction, for example clockwise, to unwind the drum cable and in the opposite direction for winding the cable around the drum. In reference to FIG. 1, the control unit 3 is in the form of a housing arranged on a bracket 31 that is assembled to the chassis 12 by a hinged linkage arm 32 for orienting the housing. A safety system is provided in order to limit the winding of the cable in the case of a dysfunction of the winch or the control unit, and thus to protect the apparatus and the person. This system comprises a plate 234, fixed to the rear end 11 of the tray, fitted with a transversal slit 234a for passing the cable. This plate serves as a stopper to a safety stud 211, the size of which is larger than that of the slit, fixed on the portion of the cable comprised between the pulley and the stopper plate.

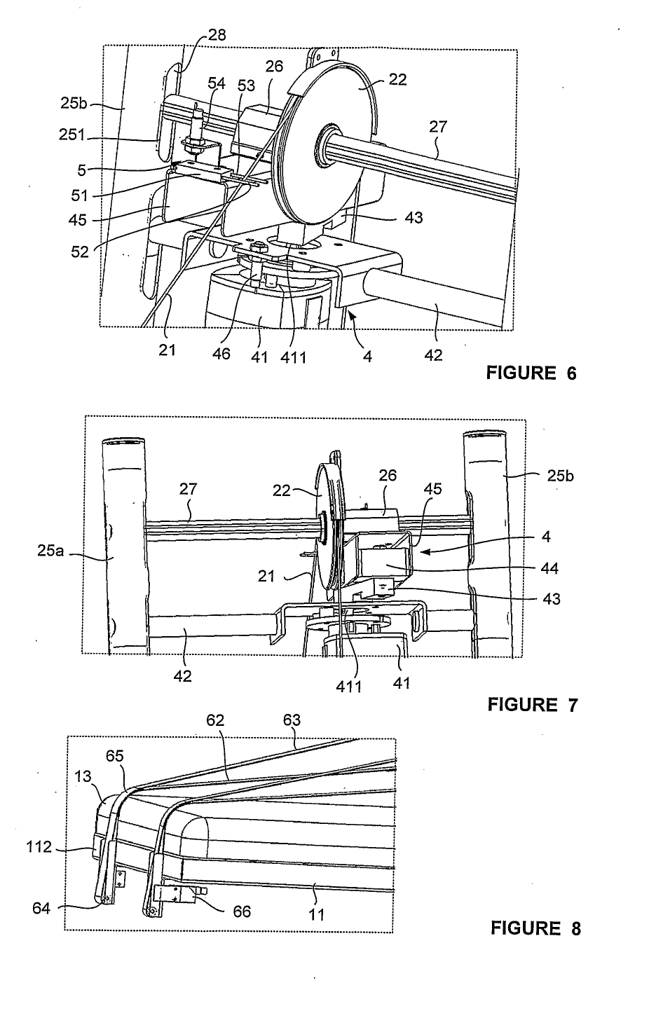

The apparatus further comprises an oscillation system 4, mounted between the posts under the pulley, able to move the pulley on its crossbar, on either side of a reference position wherein the pulley is arranged substantially according to the table's longitudinal plane, equidistant to the two posts. With reference to FIGS. 6 and 7, the oscillation system comprises a so-called oscillation motor 41, assembled to a supporting crossbar 42 mounted under the pulley between the two posts, the drive shaft 411 of which is arranged generally parallel to the posts. A cam connecting rod 43 is fixed to the shaft and bears at its free end a parallelepiped cam shoe 44. The cam shoe is pivotally mounted on the connecting rod, around a vertical axis, and sliding in a guiding rail 45 of a U-shaped cross-section that is secured to the slider and perpendicularly arranged to the splined crossbar. Rotation of the shoe 44 by the oscillation motor 41 brings about a back-and-forth movement of the pulley on either side of its reference position. The motor's shaft is off axis with respect to the longitudinal plane such that the pulley moves along an identical distance on either side of its reference position when the shoe undertakes a complete rotation. The motor is rotationally controlled by the control unit. The control unit is connected to a position sensor 46 to ensure the stopping of the pulley in its reference position after an oscillation phase. In the illustrated embodiment, this position sensor is mounted on the supporting crossbar 42 and makes it possible to detect a position of the cam connecting rod 43 corresponding to the pulley's reference position.

An angle sensing system 5 makes it possible to detect the angle of the cable downstream from the pulley, and thus the angle of the legs connected to the cable with respect to the horizontal. This sensing system comprises a stem support 51, pivotally mounted on the rail 45 around a vertical axis, which bears a front horizontal stem 52 and a rear horizontal stem 53 wherebetween the cable passes. A position sensor 54 makes it possible to detect a reference position of the stem support in which the stems are arranged parallel to the crossbars 42 and 27, this reference position corresponding to the sought cable angle. This sensor is connected to the control unit for controlling the winch motor.

To make the cable angle vary, the pulley and its associated oscillation system are height-adjustable between the two posts. For this end, with reference to FIG. 6, the posts 25a, 25b are formed of hollow tubes, the crossbar of the pulley 27 and the supporting crossbar 42 of the oscillation motor pass through the longitudinal slits 251 of the posts and are end-assembled to tubes or mounting plates 28 that slide in the posts. Blocking means such as a screw system 29 (FIG. 4), enable the blocking of the mounting tubes in the posts at various heights. Alternatively, the height adjusting operation of the pulley can be automated, for example by means of lifting jacks mounted in the posts for moving the mounting tubes.

The table is further equipped with a strapping system 6 for holding a lying down person from its armpits. With reference to FIGS. 1 and 6, this strapping system comprises two rings 61 for being slipped on through the arms to the armpits, each ring is connected to a first and a second strap 62, 63. The first strap 62 passes over a return pulley 64 fixed on the front edge 112 of the tray, and the tightening of both straps is achieved by attaching the free ends of the two straps together by a fastening system 65 of a hook and loop type, for example. To make sure that the person is properly attached before starting a control cycle of the winch motor and the oscillation motor, as well as during a control cycle, the apparatus advantageously comprises means, connected to the control unit, for detecting that the straps are actually in a tightened position. By way of example, these strap-tightening sensing means comprise contactors against which the first straps 62 in tightened position abut.

As illustrated in FIG. 1, the winch is protected by a detachable lower casing 71 fitted with a transversal slit (not shown) on the upper face for passing the cable. The upper pulley and the oscillation system are housed in an upper casing 72, its rear face 721 being fitted with a transversal slit (not shown), and a T-shaped slit on its front face 722, referenced 721a, for passing the cable during the lifting phases and oscillation phases of the legs as described hereinafter.

The operation of the apparatus according to the invention is as follows. The person lies down on the table with the pelvis in a defined reference position by pressing the buttocks against the mattress' bearing surface 132. The cable 21 is fixed to the person's feet via the chutes 241, the cable being sufficiently unwound from the winch for tightening the chutes around the ankles. The person is attached by placing the rings 61 at the armpits and by tightening the straps 62, 63.

The control unit 3 is then activated, for example by operating an activation button in order to start a preprogrammed control cycle. The pulley is initially in its reference position, substantially according to the axis of the vertebral column of the lying person. At first, the control unit controls the winch motor 231 for winding the cable and thus, effecting a traction on the legs in order to lift them. As long as the cable does not reach the cable angle corresponding to the desired slope of the legs, the cable abuts against the rear stem 53 of the angle sensor 5, thus maintaining the stem support 51 away from its reference position. When approaching the sought angle, the cable abuts against the front stem 52. Upon detection by the sensor of the bracket being in its reference position, the control unit stops the winch motor. After this phase of leg lifting, the control unit triggers an oscillation phase in which it controls the start up of the oscillation motor 41 for a determined period in order to effect movements of lateral oscillation of both legs in order to relax the pelvis and lumbar region. At the end of this oscillation phase, the control unit controls the stopping of the oscillation motor as soon as the position sensor 46 detects the cam connecting rod 43, such that the pulley is in the table's axis and thus, according to the axis of the vertebral column.

The control unit then triggers a traction phase that consists in controlling the winch motor for effecting an additional traction on the legs, in the axis of the column, in order to stretch the column, and more particularly the lower back. The winch motor is stopped, the traction is maintained for a determined period then the control unit controls the motor in the opposite direction for unwinding the cable in order to stop this additional traction. The control unit then controls the winch motor for unwinding the cable in order to bring the legs in abutment against the mattress.

If a lack of tightening is detected by one of the tightening sensors 66 during a control cycle, the control unit suspends the cycle and controls the winch for unwinding the cable.

According to another type of control cycle, after the lifting phase and the oscillation phase, the control unit triggers a so-called traction/oscillation phase in which the control unit controls the winch for an additional traction by maintaining the oscillation motor activated for keeping the movements of lateral oscillation of the legs during the stretching, this traction/oscillation phase is maintained for a determined period and is possibly repeated after a new oscillation phase.

According to other control cycles, the control unit triggers a number of traction phases or successive traction/oscillation phases, possibly between two oscillation phases, or triggers a number of successive sequences of oscillation phases followed by traction or traction/oscillation phases.

The control unit can comprise various preprogrammed cycles, selected for example by activating external buttons. Each cycle can be adjusted to vary within parameters: the speed of the winch's rotation; the activating time of the winch during the traction phase or the traction/oscillation phase, i.e. the length of the wound cable and thus the traction force; the traction time; the speed of the motor and thus, the oscillation frequency; the duration of oscillations. The cycles can be manually parametered by means of external control buttons.

By way of example, the height of the pulley will be adjusted to vary the slope of the legs between 20 and 50 degrees with respect to the horizontal, for example of about 40 degrees, the oscillation phase is maintained for 10 to 60 seconds, the amplitude of oscillations is for example a of 20 degrees on either side of the column's axis, the motor's rotational speed (oscillation frequency) is comprised between 40 and 120 rpm, the traction phase or traction/oscillation phase is maintained for 1 to 30 seconds.

The apparatus could comprise a set of cam connecting rods of different lengths making it possible to vary the oscillation amplitude from 5 to 30 degrees on either side of the column.

Further, the control cycles can be automatically selected and/or adjusted with parameters based on the person's data inputted to the control unit, such as the age, gender, height and weight.

According to alternative embodiments, the apparatus can comprise oscillating means not acting on the pulley but rather, acting directly on a portion of the cable located between the pulley and the person's feet. In the present embodiment, the table is stationary and the traction system serves as means for lifting the legs. Alternatively, the apparatus comprises a table including a mobile rear part for inclining the legs with respect to the vertebral column, the pulley and the oscillation system being mounted at the end of this rear part.

According to an alternative embodiment, the traction system comprises two cables each passing on an upper pulley, the two pulleys being transversally spaced apart from each other on the crossbar. Both pulleys are mounted in a freely rotating manner on a same slider, the latter being slidingly mounted on the crossbar and cooperating with an oscillation system for moving both pulleys together in a back-and-forth movement. Each cable is connected by a first end to a winch, a single winch being usable for both cables. In this last case, the first ends of the cables are advantageously fixed to a bar that is connected to the single winch by a third cable. The second ends of the cables are fixed to the ends of a rigid bar, the spacing between the fixing points of the cables on the bar being substantially equal to the spacing between the two pulleys. Fixing the legs to the bar is achieved by means of two leg-receiving chutes that will be assembled to the bar substantially perpendicular thereto, between the two fixing points of the cables. When the apparatus comprises one single cable, a rocking movement of the legs around the fixing point of the cable may occur during the oscillation phases. This two-cable apparatus makes it possible to limit or prevent this leg-rocking phenomenon.

According to another more compact mounting, each previously-described pulley for the passage of a cable is replaced by two pulleys, of smaller diameter, spaced apart from each other in the apparatus' longitudinal direction. Both pulleys are mounted as before on the slider, and are arranged on either side of the crossbar.

Although the invention has been described in connection to a particular embodiment, it is to be understood that it is in no way limited thereto and that it includes all the technical equivalents of the means described as well as their combinations should these fall within the scope of the invention.

1. An apparatus for stretching the vertebral column of a person, particularly the lower back, comprising a table for supporting a person lying on the back, which table is provided with a traction system including at least one cable, fixing means for connecting said cable to the feet of the person as well as traction means acting on the cable for effecting a traction on the legs in order to stretch the legs and the vertebral column, the apparatus further comprising oscillating means acting on said cable so as to obtain movements of lateral oscillation of both legs on either side of the axis of the person's vertebral column, and a control unit adapted to control said traction means and said oscillating means for combining movements of lateral oscillation of the legs and traction movements on the legs.

2. The apparatus according to claim 1, wherein the traction system comprises at least one pulley, mounted on a crossbar that is transversally assembled to the table, and whereon passes said cable, said oscillating means adapted to transversally move said pulley on said crossbar in order to effect said movements of lateral oscillation.

3. The apparatus according to claim 2, wherein said oscillating means comprise a cam system fitted between the drive shaft of a motor and said pulley for transforming the rotational movement of the shaft into a back-and-forth movement of the pulley on either side of the longitudinal plane of the table, said motor being rotationally controlled by said control unit.

4. The apparatus according to one claim 1, further comprising lifting means for lifting the legs at an angle determined with respect to the axis of the column during the oscillation movements and the traction movement(s).

5. The apparatus according to claim 2, wherein the traction system comprises two posts fixed to the table and supporting on the upper part of said posts said crossbar above the table, said traction means thus serving as leg lifting means.

6. The apparatus according to claim 5, further comprising an angle sensing system for detecting the angle of the cable between the pulley and the fixing means, said sensing means being connected to the control unit for controlling the traction means and the oscillating means.
